(19) United States
(12) Patent Application Publication
(10) Pub. No.: US 2026/0137547 A1
(43) Pub. Date: May 21, 2026

(54) FLUID COLLECTION DEVICES HAVING MULTIPLE FLUID COLLECTION REGIONS, AND RELATED SYSTEMS AND METHODS

(71) Applicant: PureWick Corporation, Covington, GA (US)

(72) Inventors: Jacob Brennan, Durham, NC (US); Camille R. Newton, Bonsall, CA (US); Raymond J. Newton, Bonsall, CA (US)

(21) Appl. No.: 19/447,347

(22) Filed: Jan. 13, 2026

Related U.S. Application Data
(63) Continuation of application No. 17/878,268, filed on Aug. 1, 2022, now Pat. No. 12,551,366.
(60) Provisional application No. 63/228,244, filed on Aug. 2, 2021.

(51) Int. Cl. A61F 5/451; A61F 5/44; A61F 5/455

(52) U.S. Cl. CPC A61F 5/451; A61F 5/4405; A61F 5/455

(57) ABSTRACT

Examples relate to fluid collection devices, and related systems and methods. In some embodiments, a fluid collection device includes a fluid impermeable barrier and a fluid permeable body. The fluid impermeable barrier may at least partially define a chamber, an opening in fluid communication with the chamber, an aperture, and multiple fluid collection regions each angled relative to one or more adjacent fluid collection regions of the multiple fluid collection regions. One or more conduits may be positioned at least partially within the chamber and extending through or in fluid communication with the aperture in the fluid impermeable barrier. At least one inlet may be in fluid communication with the one or more conduits and positioned or positionable in each of the multiple fluid collection regions. The fluid permeable body may be positioned within the chamber and extend across at least a portion of the opening.

### CROSS-REFERENCE TO RELATED APPLICATIONS

This application is a continuation of U.S. Application No. 17/878,268 filed on August 1, 2022, which claims priority to U.S. Provisional Patent Application No. 63/228,244 filed on August 2, 2021, the disclosure of each of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters may be uncomfortable, painful, and may cause urinary tract infections.

Thus, users and manufacturers of fluid collection devices continue to seek new and improved devices, systems, and methods to collect urine.

### SUMMARY

Embodiments disclosed herein are related to fluid collection devices, and related systems and methods. In an embodiment, a fluid collection device includes a fluid impermeable barrier at least partially defining a chamber, an opening in fluid communication with the chamber, an aperture, and multiple fluid collection regions each angled relative to one or more adjacent fluid collection regions of the multiple fluid collection regions. The fluid collection device also includes one or more conduits positioned at least partially within the chamber and extending through or in fluid communication with the aperture in the fluid impermeable barrier. The fluid collection device also includes at least one inlet in fluid communication with the one or more conduits and positioned or positionable in each of the multiple fluid collection regions. The fluid collection device also includes a fluid permeable body positioned within the chamber and extending across at least a portion of the opening.

In an embodiment, a method of collecting fluid includes positioning an opening defined at least partially by a fluid impermeable barrier of a fluid collection device at least proximate to a region of a user from which fluid is to be withdrawn. The fluid impermeable barrier at least partially defines a chamber, an aperture, and multiple fluid collection regions. The fluid collection device includes a fluid permeable body extending at least partially across the opening, one or more conduits positioned at least partially within the chamber and extending through or in fluid communication with the aperture, and at least one inlet in fluid communication with the one or more conduits and positioned or positionable in each of the multiple fluid collection regions. The method also includes adjusting the fluid collection device to provide fluid communication between the aperture and the at least one inlet in a first fluid collection region of the multiple fluid collection regions and inhibiting fluid (e.g., vacuum) communication between the aperture and a second fluid collection region of the multiple fluid collection regions. The first fluid collection region is positioned lower than at least the second fluid collection region such that gravity draws fluid to the first fluid collection region. The method also includes collecting fluid from the user in the first fluid collection region. The method also includes drawing the fluid from the first fluid collection region through the conduit and through the aperture.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is a block diagram of a fluid collection system, according to an embodiment.

FIG. 2A is a front isometric view of a fluid collection device, according to an embodiment.

FIG. 2B is a front isometric view of the fluid collection device of FIG. 2A with a fluid permeable body of the fluid collection device removed.

FIG. 2C is a rear view of the fluid collection device of FIG. 2A.

FIG. 2D is a cross-sectional view of the fluid collection device of FIG. 2A taken along plane 2D-2D of FIG. 2B and with the fluid permeable body removed.

FIG. 3 is a cross-sectional view of a portion of a fluid collection device, according to an embodiment.

FIG. 4A is a front isometric view of a fluid collection device, according to an embodiment.

FIG. 4B is a front isometric view of the fluid collection device of FIG. 4A with a fluid permeable body of the fluid collection device removed.

FIG. 4C is a rear view of the fluid collection device of FIG. 4A.

FIG. 4D is a cross-sectional view of the fluid collection device taken along plane 4D-4D of FIG. 4B and with the fluid permeable body removed.

FIG. 4E is a cross-sectional view of the fluid collection device taken plane 4E-4E of FIG. 4B and with the fluid permeable body removed.

FIG. 5 is a flow diagram of a method for collecting fluid, according to an embodiment.

### DETAILED DESCRIPTION

Embodiments disclosed herein are related to fluid collection devices multiple fluid collection regions, and related systems and methods. The devices and systems disclosed herein are configured to collect fluids from an individual. The fluids collected by the fluid collection devices may include at least one of urine, vaginal discharge, penile discharge, reproductive fluids, blood, sweat, wound exudate, or other bodily fluids. Conventional fluid collection devices typically include a single fluid collection region and one conduit (e.g. , tube) having an inlet positioned proximate to the single fluid collection region. Conventional fluid collection devices, then, are limited in the directions the fluid collection devices may be used because a single fluid collection region typically may be at the low point of the fluid collection device such that gravity pulls fluid to the fluid collection region. This limitation may result in the fluid collection device being less efficient and less effective in certain positions of use.

Embodiments of fluid collection devices described herein include multiple fluid collection regions and one or more conduits configured to draw fluid from each of the multiple fluid collection regions. The multiple fluid collection regions, then, may allow the fluid collection device to be effective for use in multiple positions of the fluid collection device and/or the user. For example, in some embodiments, the fluid collection device may include multiple fluid collection regions spaced around an axis of the fluid collection device such that one of the fluid collection regions may always be position at or proximate to the low point of the fluid collection device. In some embodiments, the fluid collection device may include a valve configured to provide fluid communication between a pump or vacuum source and only the lower fluid collection region of the multiple fluid collection regions. Fluid communication may include any combination of liquid and/or gaseous communication, such as vacuum communication with the vacuum source.

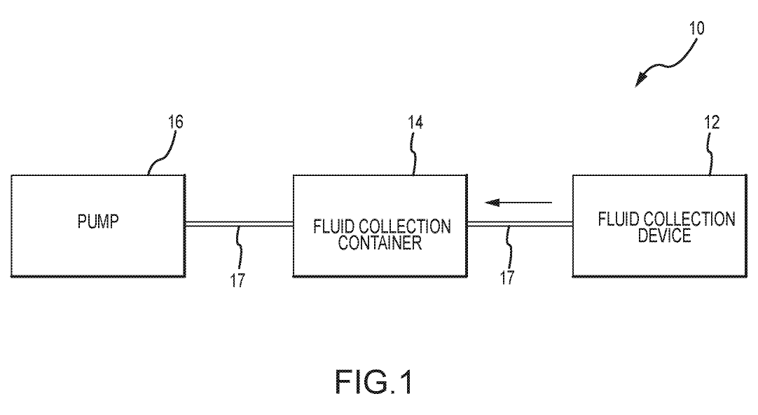

FIG. 1 is a block diagram of a fluid collection system 10, according to an embodiment. The fluid collection system 10 may be included in embodiments of fluid collection systems described herein. The system 10 includes a fluid collection device 12 (e.g., any of the fluid collection devices disclosed herein), a urine collection container 14, and a pump 16 (or vacuum source). The fluid collection device 12, the urine collection container 14, and the pump 16 may be fluidly coupled to each other via one or more conduits 17. For example, fluid collection device 12 may be operably coupled to one or more of the urine collection container 14 or the pump 16 via the conduit 17. In some embodiments, the pump 16 may be secured directly to the urine collection container 14. Fluid (e.g., urine or other bodily fluids) collected in the fluid collection device 12 may be removed from the fluid collection device 12 via the conduit 17 secured to the fluid collection device 12. Suction force may be introduced into the chamber of the fluid collection device 12 via the inlet of the conduit 17 responsive to suction (e.g., vacuum) force applied at the outlet of the conduit 17.

The suction force may be applied to the outlet of the conduit 17 by the pump 16 either directly or indirectly. The suction force may be applied indirectly via the urine collection container 14. For example, the outlet of the conduit 17 may be disposed within or fluidly coupled to an interior region of the urine collection container 14 and an additional conduit 17 may extend from the urine collection container 14 to the pump 16. Accordingly, the pump 16 may apply suction to the fluid collection device 12 via the urine collection container 14. The suction force may be applied directly via the pump 16. For example, the outlet of the conduit 17 may be disposed within the pump 16. An additional conduit 17 may extend from the pump 16 to a point outside of the fluid collection device 12, such as to the urine collection container 14. In such examples, the pump 16 may be disposed between the fluid collection device 12 and the urine collection container 14.

The urine collection container 14 is sized and shaped to retain a fluid therein. The urine collection container 14 may include a bag (e.g., drainage bag), a bottle, a canister, or a cup (e.g., collection jar), or any other enclosed container for storing bodily fluid(s) such as urine. In some examples, the conduit 17 may extend from the fluid collection device 12 and attach to the urine collection container 14 at a first point therein. An additional conduit 17 may attach to the urine collection container 14 at a second point thereon and may extend and attach to the pump 16. Accordingly, a vacuum (e.g., suction) may be drawn through fluid collection device 12 via the urine collection container 14. Fluid, such as urine, may be drained from the fluid collection device 12 using the pump 16.

The pump 16 may include one or more of a manual vacuum pump, and electric vacuum pump, a diaphragm pump, a centrifugal pump, a displacement pump, a magnetically driven pump, a peristaltic pump, or any pump configured to produce a vacuum. The pump 16 may provide a vacuum or suction to remove fluid from the fluid collection device 12. In some examples, the pump 16 may be powered by one or more of a power cord (e.g., connected to a power socket), one or more batteries, or even manual power (e.g., a hand operated vacuum pump). In some examples, the pump 16 may be sized and shaped to fit outside of, on, or within the fluid collection device 12. For example, the pump 16 may include one or more miniaturized pumps or one or more micro pumps. The vacuum sources disclosed herein may include one or more of a switch, a button, a plug, a remote, or any other device suitable to activate the pump 16.

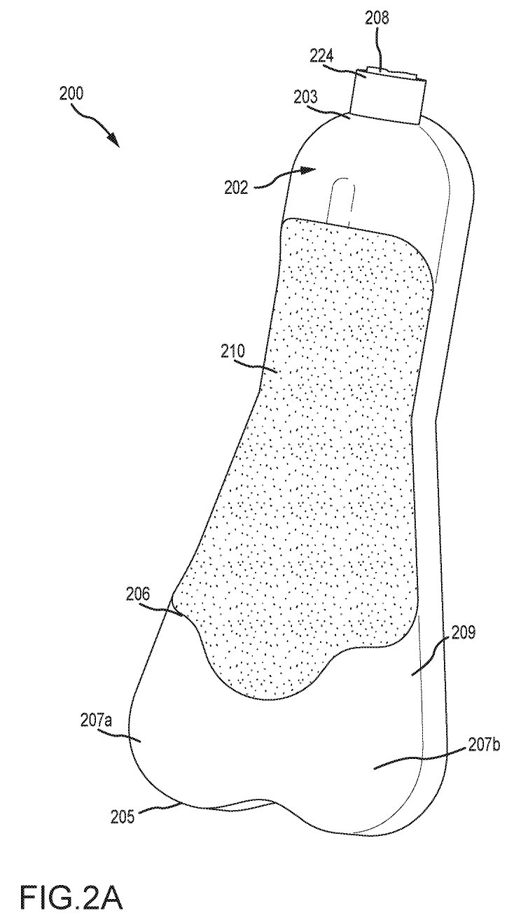

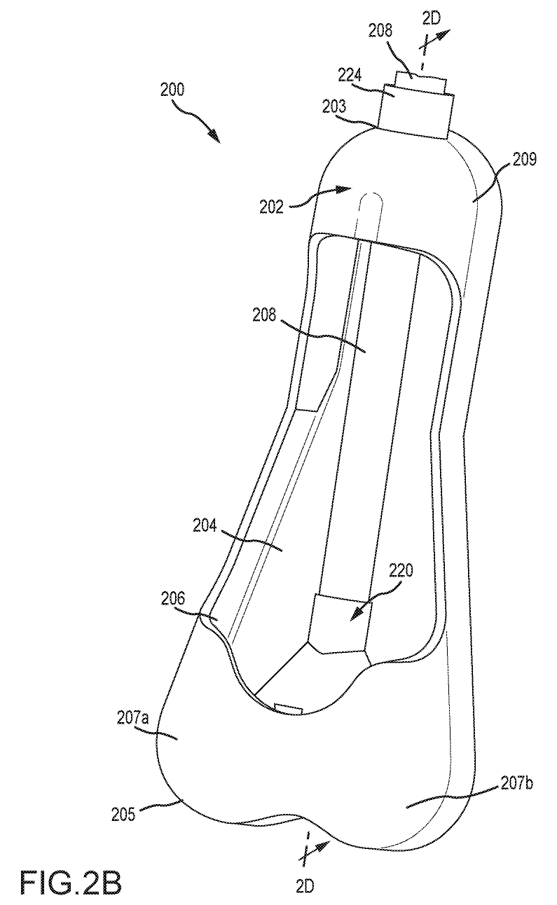

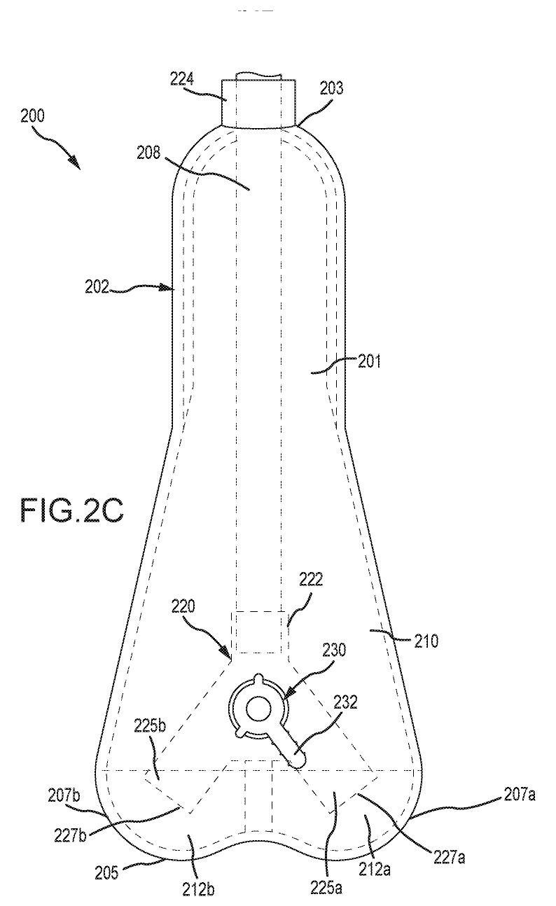

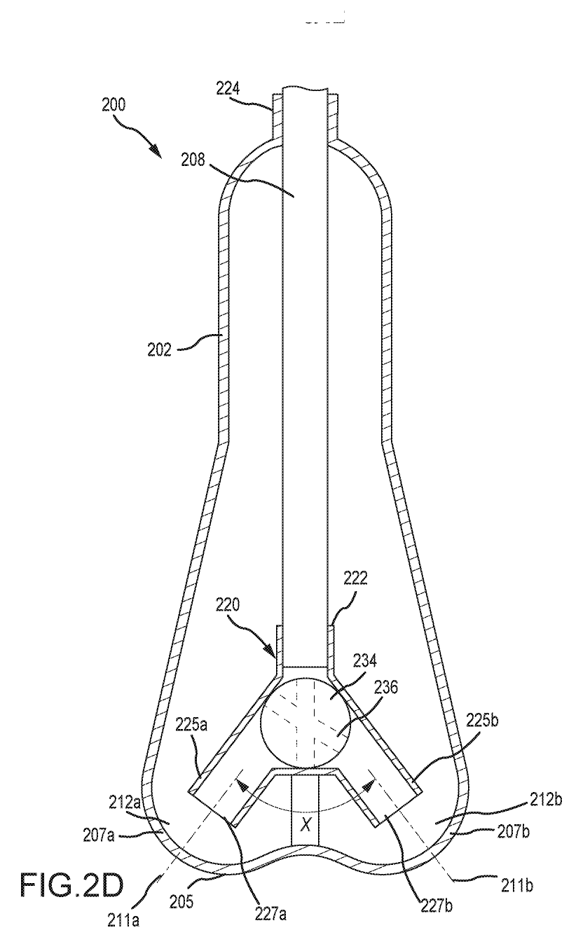

FIG. 2A is a front isometric view of a fluid collection device 200 having a fluid impermeable barrier 202 with multiple fluid collection regions 207a, 207b and a fluid permeable body 210. FIG. 2B is a front isometric view of the fluid collection device 200 with the fluid permeable body 210 removed, FIG. 2C is a rear view of the fluid collection device 200, and FIG. 2D is a cross-sectional view of the fluid collection device 200 taken along plane 2D-2D of FIG. 2B. The fluid collection device 200 is an example of a fluid collection device that is configured to receive urine from a female or a male, and/or collect other fluids such as vaginal discharge, penile discharge, reproductive fluids, blood, sweat, wound exudate, or other bodily fluids from a user . The fluid collection device 200 is configured to draw urine or other fluids away from the urethral opening, skin, and/or wound of the user for temporary collection in one or more of the fluid collection regions 207a, 207b before the fluid is withdrawn from the fluid collection device through a conduit 208.

The fluid collection device 200 includes a fluid impermeable barrier 202 having a distal end region 205, a proximal end region 203, a front region 209 at least partially defining an opening 206, and a back (e.g., rear) region 201. The front region 209 may interface the region of the body of the user from which fluids are to be collected, and the back region 201 may be oriented away from the region of the body of the user from which fluids are to be collected. In some usages, the front region 209 may be oriented at least partially upwards such that urine discharged by a user falls onto the opening 206 for collection. The fluid impermeable barrier 202 at least partially defines a chamber 204 (e.g., interior region, shown in FIG. 2B) and includes an inward border or edge defining an opening 206 on the front region 209. The fluid permeable body 210 may be positioned at least partially within the chamber 204 and extend across at least a portion (e.g., all) of the opening 206.

The back region 201 may be substantially planar and the front region 209 may be at least partially curved, arced, or convex, according to some embodiments. In some embodiments, at least a portion of the fluid impermeable barrier 202 and the fluid permeable body 210 may be substantially cylindrical. For example, a proximal portion of the fluid impermeable barrier 202 may be substantially cylindrical until the fluid collection regions 207a, 207b begin. The fluid impermeable barrier 202 may by substantially or somewhat Y-shaped, with the proximal end region 203 being the single end of the Y shape and the distal end region 205 being the two-pronged end of the Y shape.

The opening 206 is formed in and extends longitudinally through the fluid impermeable barrier 202, thereby enabling fluids to enter the chamber 204 from outside of the fluid collection device 200. The opening 206 includes a region of the fluid collection device 200 where the fluid impermeable barrier 202 is absent and does not cover the fluid permeable body 210. The opening 206 may be configured to be positioned at least proximate to (e.g., adjacent to) the opening of a female urethra, over or under the opening of a urethra of a male, over or in a wound of a user, and/or on the skin of user. The opening 206 may exhibit an elongated shape that is configured to extend from a first location below the urethral opening (e.g., at or near the anus or the vaginal opening) to a second location above the urethral opening (e.g., at or near the clitoris or the pubic hair). The opening 206 may exhibit an elongated shape since the space between the legs of a female is relatively small when the legs of the female are closed, thereby only permitting the flow of the fluids along a path that corresponds to the elongated shape of the opening 206. For example, the opening may extend longitudinally along the fluid impermeable barrier. The opening 206 in the fluid impermeable barrier 202 may exhibit a width that is measured transverse to the longitudinal direction and may be at least about 10% of the circumference of the fluid collection device 100, such as about 25% to about 50%, about 40% to about 60%, about 50% to about 75%, about 65% to about 85%, or about 75% to about 100% of the circumference of the fluid collection device 200. The opening 206 may exhibit a width that is greater than 50% of the circumference of the fluid collection device 200 since the vacuum (e.g., suction) through the conduit 208 pulls the fluid into the conduit 208. In some embodiments, the opening 206 may be vertically oriented (e.g., having a major axis parallel to the longitudinal axis of the device 200). In some embodiments (not shown), the opening 206 may be horizontally oriented (e.g., having a major axis perpendicular to the longitudinal axis of the device 200). In some embodiments, the inward border or edge of the fluid impermeable barrier 202 defines the opening 206. The edge may include two or more opposing arced portions, the arced portions following the outer circumference or periphery of the fluid impermeable barrier 202. In some embodiments, the opening 206 widens between the proximal end region 203 and the distal end region 205 such that the opening 206 is more wide near the distal end region 205 than the proximal end region 203. In some embodiments, the front region 209 of the fluid impermeable barrier 202 includes a concave edge defining a portion of the opening 206 between the two fluid collection regions 207a, 207b.

The fluid impermeable barrier 202 also may define an aperture 224 (e.g., port) sized to receive the conduit 208 (e.g., at least one tube). The aperture 224 may be positioned at the proximal end region 203 of the fluid impermeable barrier 202 and may be sized and dimensioned to have the conduit 208 removably or fixedly secured thereto or extending therethrough. The at least one conduit 208 may be disposed in the chamber 204 via the aperture 224. The aperture 224 may be configured to form an at least substantially fluid tight seal against the conduit 208 or the at least one tube thereby substantially preventing the fluids from escaping the chamber 204. In some embodiments, a seal separate from the fluid impermeable barrier 202 may seal the aperture 224 with the conduit 208 extending therethrough.

The fluid collection device 200 also includes conduit 208 that is at least partially disposed in the chamber 204. The conduit 208 (e.g., a tube) may be referred to herein as a proximal conduit 208 and may include an inlet secured or securable to a proximal port 222 of a manifold 220 positioned within the chamber 204 and/or secured to the fluid impermeable barrier 202. The conduit 208 provides fluid communication between an interior region of the chamber 204 (via the manifold 220) and a fluid storage container (not shown) or a portable vacuum source (not shown). For example, the conduit 208 may directly or indirectly fluidly couple the interior region of the chamber 204 with the fluid storage container or the portable vacuum source.

The fluid impermeable barrier 202 may also temporarily store the fluids in the chamber 204. For example, the fluid impermeable barrier 202 may be formed of any suitable fluid impermeable materials, such as a fluid impermeable polymer (e.g., silicone, polypropylene, polyethylene, polyethylene terephthalate, a polycarbonate, etc.), polyurethane films, thermoplastic elastomer (TPE), rubber, thermoplastic polyurethane, another suitable material, or combinations thereof. The fluid impermeable barrier 202 is flexible, thereby enabling the fluid collection device 100 to bend or curve when positioned against the body of a wearer. Example fluid impermeable barriers may include, but are not limited to, a fluid impermeable barrier including at least one of Versaflex CL 2000X TPE, Dynaflex G6713 TPE, or Silpuran 6000/05 A/B silicone. In an embodiment, the fluid impermeable barrier 202 may be air permeable. In such an embodiment, the fluid impermeable barrier 202 may be formed of a hydrophobic material that defines a plurality of pores. In an embodiment, one or more portions of at least the outer surface of the fluid impermeable barrier 202 may be formed from a soft and/or smooth material, thereby reducing chaffing.

The fluid collection regions 207a, 207b may be at least partially (e.g., entirely) defined by the fluid impermeable barrier 202 generally opposite to the proximal end region 203 and at the distal end region 205. The fluid collection device 200 may include multiple fluid collection regions each angled relative to one or more adjacent fluid collection regions of the multiple fluid collection regions. For example, although the fluid collection device 200 shown in FIGS. 2A-2D includes two fluid collection regions 207a, 207b, the fluid collection device may include three, four, five, six, etc., fluid collection regions each angled relative to one or more adjacent fluid collection regions of the multiple fluid collection regions. In some embodiments, the fluid collection device 200 may be generally ψ- or +-shaped and include a single proximal end 203 and three fluid collection regions at the distal end region 205 of the fluid impermeable barrier 202. The fluid collection regions of embodiments having three or more fluid collection regions may include any aspect of the fluid collection regions 207a, 207b described herein.

The fluid collection regions 207a, 207b may be angled relative to one or more adjacent fluid collection regions 207a, 207b. For example, turning to FIG. 2D, each of the fluid collection regions 207a, 207b may include an approximate center or axis 211a, 211b. The center or axis 211a, 211b of the fluid collection region 207a, 207b may be angled X degrees from the center or axis 211a, 211b of the adjacent fluid collection region 207a, 207b. In some embodiments, the center or axis 211a, 211b of the fluid collection regions 207a, 207b may be angled from the center or axis 211a, 211b of the adjacent fluid collection region 207a, 207b about 10 degrees to about 135 degrees, about 10 degrees to about 90 degrees, about 10 degrees to about 45 degrees, about 45 degrees to about 90 degrees, about 90 degrees to about 135 degrees, about 10 degrees to about 30 degrees, about 30 degrees to about 50 degrees, about 50 degrees to about 70 degrees, about 70 degrees to about 90 degrees, about 90 degrees to about 110, about 110 degree to about 130 degrees, at least about 10 degrees, at least about 20 degrees, at least about 30 degrees, at least about 40 degrees, at least about 50 degrees, at least about 60 degrees, at least about 70 degrees, at least about 80 degrees, at least about 90 degrees, at least about 100 degrees, at least about 110 degrees, at least about 120 degrees, or at least about 130 degrees.

Each of the fluid collection regions 207a, 207b of the fluid impermeable barrier 202 may at least partially define a reservoir 212a, 212b (e.g., sump) in the chamber 204 for fluid collection or storage. In some embodiments, the reservoir 212a, 212b may be an at least partially (e.g., entirely) unoccupied portion of the chamber 204 and is at least partially (e.g., entirely) void of other material. In some embodiments, the reservoir 212a, 212b is defined at least partially by a terminating end the fluid permeable body 210 (shown in FIG. 2C) and the fluid collection regions 207a, 207b at the distal end region 205 of the fluid impermeable barrier 202. In some embodiments, the reservoirs 212a, 212b are substantially filled with one or more of the fluid permeable materials described herein.

The fluid collection device 200 also may include the manifold 220 or other conduit splitting or dividing element positioned within the chamber 204. The manifold 220 may include the proximal port 222 configured to fixedly or detachably secure to the inlet of the conduit 208. In some embodiments, the manifold 220 is integrally formed (e.g. , extruded or molded) with the conduit 208, and the proximal port 222 is absent. The manifold 220 also includes at least two arms 225a, 225b extending at least partially into a different one of the two fluid collection regions 207a, 207b. In fluid collection device 200 having more than two fluid collection regions 207a, 207b, the manifold 220 may include more than two arms 225a, 225b. In some embodiments, the manifold 220 includes a number of arms equal to the number of fluid collection regions on the fluid collection device 200. For example, a fluid collection device 200 having three fluid collection regions may include three arms each extending at least partially into a different one of the three fluid collection regions. A fluid collection device 200 having four fluid collection regions may include four arms each extending at least partially into a different one of the four collection regions. In some embodiments, the arms 225a, 225b are angled the adjacent arm 225a, 225b at an angle substantially equal to the angle between the centers or axes 211a, 211b of the fluid collection regions 207a, 207b.

Each of the arms 225a, 225b includes an inlet 227a, 227b positioned at least partially within a different one of the reservoirs 212a, 212b in the fluid collection regions 207a, 207b. In some embodiments, the inlets 227a, 227b of the arms 225a, 225b may be selectively in fluid communication with proximal port 222 and/or the conduit 208. For example, the fluid collection device 200 may include a valve 230 configured to selectively allow fluid (e.g., vacuum) communication between the conduit 208 and one of the inlets 227a, 227b. The valve 230 may be valve positioned between the conduit 208 and the inlets 227a, 227b. For example, the valve 230 may be positioned at an intersection of the two arms 225a, 225b and the proximal port 222 and/or the conduit 208. The valve 230 may be selectively adjustable between a first position and at least a second position. In the first position, the valve 230 may be configured to provide fluid communication between the first inlet 227a and the conduit 208 while preventing fluid communication between the second inlet 227b and the conduit 208. In the second position, the valve 230 may be configured to provide fluid communication between the second inlet 227b and the conduit 208 while preventing fluid communication between the conduit 208 and the first inlet 227a.

In some embodiments, the valve 230 includes a ball, a cylinder, or other rotatable element 234 selectively rotatable within the manifold 220. The rotatable element 234 may include one or more fluid paths 236 extending therethrough and arranged to selectively provide fluid communication between the proximal port 222 and only one of the inlets 227a, 227b. For example, the rotatable element 234 may be selectively rotatable between a first position and a second position. In the first position, the one or more fluid paths 236 of the rotatable element 234 may be arranged to provide fluid communication between the first inlet 227a and the conduit 208 while preventing fluid communication between the second inlet 227b and the conduit 208. In the second position, the one or more fluid paths 236 of the rotatable element 234 may be arranged to provide fluid communication between the second inlet 227b and the conduit 208 while preventing fluid communication between the conduit 208 and the first inlet 227a. In embodiments of the fluid collection device 200 having three or more fluid collection regions, the valve 230 may be configured to selectively provide fluid communication between the conduit 208 and the inlet in only one fluid collection region while preventing fluid communication between the conduit 208 and the inlets in the remaining fluid collection regions. In some embodiments, the valve 230 may include an additional position that allows fluid communication between the conduit 208 and the inlets 227a, 227b of two or more adjacent fluid collection regions 207a, 207b.

The fluid collection device 200 also may include an actuator 232 (e.g., handle, dial, lever, or grip) positioned outside the chamber 204 and secured to the valve 230. The actuator is configured to adjust the valve 230 between the first position and the second position. In some embodiments, the actuator 232 may be positioned on the back region 201 of the fluid permeable barrier 202. The actuator 232 outside the chamber 204 may align with the fluid collection region 207a, 207b to which fluid communication is provided in the chamber 204. For example, the actuator 232 may align with or be positioned over the first fluid collection region 207a outside the chamber 204 when the valve 232 provides fluid communication with the first inlet 227a in the chamber 204, and the actuator 232 may align with or be positioned over the second fluid collection region 207b outside the chamber 204 when the valve 230 provides fluid communication with the second inlet 227b inside the chamber 204.

In use, the fluid collection device 200 may be positioned on the user with the opening 206 at least proximate to (e.g. , positioned against, adjacent to, or at least partially within) the region of the user from which fluid is to be collected. Depending on the position of the user and/or the fluid collection device 200, one of the first fluid collection region 207a or the second fluid collection region 207b may be positioned lower than the other such that fluid collected in the chamber flows to the lower fluid collection region. The user or a caregiver may then adjust the actuator 232 to rotate the valve 230 and provide fluid communication between the conduit 208 and the inlet in the lower fluid collection region of the fluid collection regions 207a, 207b while preventing fluid communication between the conduit 208 and the inlet(s) in the one or more higher fluid collection regions of the fluid collection regions 207a, 207b. A negative pressure or vacuum may then draw fluid in the reservoir of the lower fluid collection region through the inlet in the lower fluid collection region, into the conduit 208 and out of the fluid collection device 200. This configuration may allow the fluid collection device 200 to be more efficiently operable in a variety of positions and on a variety of locations on the user.

As noted above, the fluid collection device 200 also includes the fluid permeable body 210 positioned within the chamber 204 and extending at least partially across the opening 206 and at least partially between the distal end region 205 and the proximal end region 203. The fluid permeable body 210 may be shaped generally complementary to the shape of the chamber 204 of the fluid impermeable barrier 202. In some embodiments, the fluid permeable body 210 fills substantially all of the chamber 204, except for the reservoirs 212a, 212b. In some embodiments, the reservoirs 212a, 212b are absent, and the fluid permeable body 210 fills substantially all of the chamber 204.

The fluid permeable body 210 can be configured to wick and/or allow transport of fluid away from the opening 206 towards the reservoirs 212a, 212b and/or inlets 227a, 227b. More specifically, the fluid permeable body 210 can be configured to wick and/or allow transport of fluid away from the opening 206 toward the lower positioned reservoir and inlet of the reservoirs 212a, 212b and inlets 227a, 227b. The fluid permeable body 210 can include any material that can wick and/or allow transport of the fluid. The permeable properties referred to herein can be wicking, capillary action, diffusion, or other similar properties or processes, and are referred to herein as “permeable” and/or “wicking.” Such “wicking” or other physical properties may exclude absorption into the fluid permeable body 210, such as not include adsorption of the bodily fluids into the fluid permeable body 210. Put another way, substantially no absorption or solubility of the bodily fluids into the material may take place after the material is exposed to the bodily fluids and removed from the bodily fluids for a time. While no absorption or solubility is desired, the term “substantially no absorption” may allow for nominal amounts of absorption and/or solubility of the bodily fluids into the fluid permeable body 210 (e.g. , absorbency), such as less than about 30 wt% of the dry weight of the fluid permeable body 110, less than about 20 wt%, less than about 10 wt%, less than about 7 wt%, less than about 5 wt%, less than about 3 wt%, less than about 2 wt%, less than about 1 wt%, or less than about 0.5 wt% of the dry weight of the fluid permeable body 210. In an embodiment, the fluid permeable body 210 may include at least one absorbent or adsorbent material.

The fluid permeable body 210 can include a one-way fluid movement fabric. As such, the fluid permeable body 210 can remove fluid from the area around the urethra, wound, and/or skin, thereby leaving the area dry. The fluid permeable body 210 can enable the fluid to flow generally towards the lower positioned reservoir and inlet of the reservoirs 212a, 212b and inlets 227a, 227b. The fluid permeable body 210 can include a porous or fibrous material, such as hydrophilic polyolefin. In some embodiments, the fluid permeable body 210 consists of or consists essentially of a porous or fibrous material, such as hydrophilic polyolefin. Examples of polyolefin that can be used in the fluid permeable body 210 include, but are not limited to, polyethylene, polypropylene, polyisobutylene, ethylene propylene rubber, ethylene propylene diene monomer, or combinations thereof. Moreover, the fluid permeable body 210 can be manufactured according to various manufacturing methods, such as molding, extrusion, or sintering. The fluid permeable body 210 can include varying densities or dimensions.

In some embodiments, the fluid permeable body 210 can include two or more layers of fluid permeable materials. For example, the fluid permeable body 210 can include a fluid permeable membrane covering or wrapped around a fluid permeable support, with both the fluid permeable membrane and the fluid permeable support being disposed in the chamber 204. The fluid permeable membrane can cover or extend across at least a portion (e.g., all) of at least the side of the fluid permeable support facing the penis of the user. The fluid permeable membrane and the fluid permeable support can be configured to wick any fluid away from the opening 206, thereby preventing the fluid from escaping the chamber 204 through the opening 206 and promoting removal of the fluid from the chamber 204 through the conduit 208. The permeable properties referred to herein can be wicking, capillary action, diffusion, or other similar properties or processes, and are referred to herein as “permeable” and/or “wicking.”

The fluid permeable membrane and the fluid permeable support also can wick and/or allow transport of the fluid generally towards the lower reservoir of the reservoirs 212a, 212b. The fluid permeable membrane can include any material that can wick the fluid. For example, the fluid permeable membrane can include fabric, such as a gauze (e.g. , a silk, linen, polymer based materials such as polyester, or cotton gauze), nylon (such as a spun nylon fibers), another soft fabric (e.g., jersey knit fabric or the like), another smooth fabric (e.g., rayon, satin, or the like), and/or one or more fluid permeable paper products. Forming the fluid permeable membrane from gauze, soft fabric, and/or smooth fabric can reduce chaffing caused by the fluid collection device 200. Other embodiments of fluid permeable membranes and fluid permeable supports are disclosed in U.S. Patent Application No. 15/612,325 filed on June 2, 2017; U.S. Patent Application No. 15/260,103 filed on September 8, 2016; U.S. Patent Application No. 15/611,587 filed on June 1, 2017; PCT Patent Application No. PCT/US19/29608, filed on April 29, 2019, the disclosure of each of which is incorporated herein, in its entirety, by this reference. In many embodiments, the fluid permeable body 210 includes a fluid permeable support including a porous spun nylon fiber structure and a fluid permeable wicking membrane including gauze at least partially enclosing the spun nylon fiber structure. For example, the fluid permeable body 210 may include a gauze or other wicking fabric positioned to contact the skin of the user through the opening 206. In some embodiments, the gauze or other wicking fabric is wrapped around a body of spun nylon fibers material and/or covering both sides of a substantially planar spun nylon fibers material. In some embodiments, the gauze or other wicking fabric covers the side of substantially planar spun nylon fibers material that is oriented towards the skin of the user.

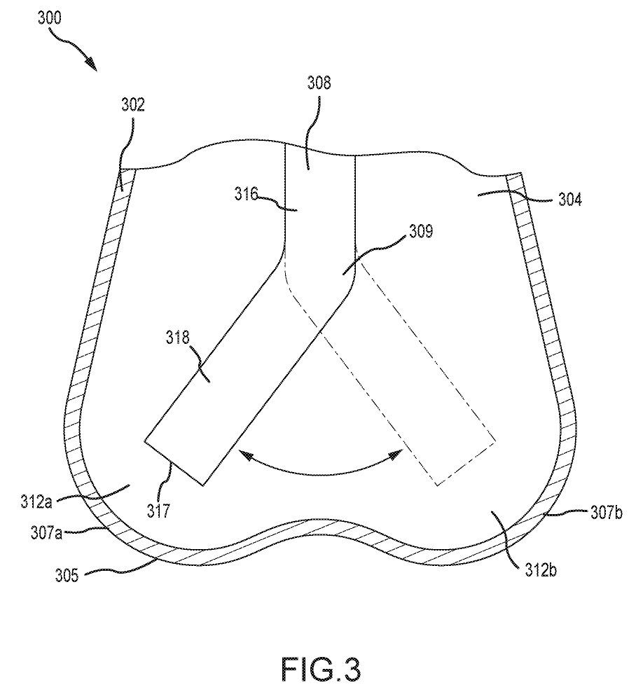

Turning now to FIG. 3, in some embodiments a fluid collection device 300 may include a single conduit 308 having an inlet 317 on a distal portion 318 that is selectively pivotable between two (or more) fluid collection regions 307a, 307b. Unless otherwise noted, the fluid collection device 300 may include any aspect of the fluid collection device 200, such as fluid impermeable barrier 302 and a chamber 304. The fluid impermeable barrier 302 and chamber 304 may include any aspect (materials, shapes, configurations, etc.) of the fluid impermeable barrier 202 and the chamber 204 of the fluid collection device 200. For example, the fluid impermeable barrier 302 may include a distal end region 305 and may at least partially define and/or include the two or more fluid collection regions 307a, 307b positioned at least proximate to the distal end region 305. Moreover, although not shown in FIG. 3 , the fluid collection device 300 may include an opening, an aperture, and fluid permeable body similar to or the same as those described in relation to the fluid collection device 200. The fluid collection device 300 also may include a reservoir 312a, 312b in each of the fluid collection regions 307a, 307b. The reservoirs 312a, 312b may include any aspect of the reservoirs 212a, 212b described above in the fluid collection device 200.

The conduit 308 may extend through the aperture at the proximal end region of the fluid impermeable barrier 302 into the chamber and towards the distal end region 305. The conduit 308 may include one inlet 317 on the terminating distal end of the one conduit 308 that is selectively adjustable between a first configuration having the inlet 317 positioned in the first fluid collection region 307a and a second configuration having the inlet 317 positioned in the second fluid collection region 307b. In some embodiments, the conduit 308 includes a proximal portion 316 that remains in substantially the same position (e.g., substantially stationary) when the conduit 308 is adjusted between the first configuration and the second configuration. The conduit 308 also may include a distal portion 318 that moves between the first fluid collection region 307a in the first configuration and the second fluid collection region 307b in the second configuration. In some embodiments, the conduit 308 also includes an adjustable joint 309 or other bendable region positioned between the proximal portion 316 and the distal portion 318. The adjustable joint 309 may include any selectively bendable joint or region configured to allow a user to bend the conduit 308 to a desired position and also retain the conduit in that position. For example, the conduit 308 may include a concertina- or accordion-type hinge. Other embodiments of pivotable fluid collection device tubing connectors that may be used as the adjustable joint 309 are disclosed in U.S. Patent Application No. 63/147,013 filed on February 8, 2021, the disclosure of which is incorporated herein, in its entirety, by this reference.

In some embodiments, at least the adjustable joint 309 of the conduit 308 may include a shape memory material such as a shape memory polymer or a metal (e.g., shape memory metal). The shape memory material may be secured to or embedded into at least a portion of the conduit 308. Suitable shape memory materials are composed to adopt an intermediate or permanent shape in response to a stimuli. The stimuli may include an external physical force (e.g. , bending force), heat, electrical bias, or a magnetic field. While the term “shape memory” is used to describe some of the “shape memory materials” herein, it should be understood that, in some examples, the material modified by the term “shape memory” may not necessarily need to return to a preselected shape upon application of a stimuli, as understood as the classical definition of the “shape memory material.” Rather, at least some of the shape memory materials herein may simply hold a selected shape when bent, set, or cured into a specific shape and/or when cooled in a specific shape, regardless of the stimuli applied thereto after. The shape memory materials may be returned to the original shape or changed to a new shape by application of stimuli. For example, a metal wire bent to a first shape may be utilized as the shape memory material, whereinafter the metal wire may be modified to a second shape via physical force applied thereto or via heating.

In an embodiment, the shape memory material may include metal, such as an elemental metal, an alloy, or shape memory alloy. Suitable shape memory metals may include standard steels, stainless steel, carbon alloy steel, head treated steel, aluminum, silver, copper, iron, nickel, zinc, tin, beryllium, or the like. Suitable shape memory alloys may include stainless steel; galvanized steel; aluminum alloys; nickel-titanium alloys, such as Nitinol, Ni-Ti-Cu, Ni-Ti, Co, or the like; copper-based alloys such as Cu-Zn-Al, Cu-Al-Ni, Cu-Al-Sn, or the like; Co-Cr-Ni-Mo alloys (e.g., Elgiloy®) or the like; or any other alloy having shape memory characteristics. As explained above, the shape memory metals or alloys may merely be metals or alloys that may be shaped to a selected configuration. In some examples, the shape memory metals or alloys may return to a primary shape when an external stimuli is applied thereto. In some examples, the outer surface of the shape memory metal may be coated with a polymer, anodized, passivated, or otherwise treated to prevent corrosion.

Shape memory polymers (“SMPs”) may include polyurethane-based SMPs such as a copolymer (e.g., copolyester, polyurethane, polyetherester, etc.) including blocks of one or more of poly(ε-caprolactone), polyethyleneterephthalate (PET), polyethyleneoxide (PEO), polyethylene glycol (PEG), polystyrene, polymethylmethacrylate (PMMA), Polybutylmethacrylate (PBMA), poly(N,N-butadiene), poly(N-methyl-N-oxazoline), polytetrahydrofuran, or poly(butylene terephthalate); thermoplastic polymers such as polyether ether ketone (PEEK), nylon, acetal, polytetrafluoroethylene (PTFE), polysulphone, or the like; Polynorbonene; other deformable polymers; or any other shape memory polymer.

In some embodiments, the fluid collection device may include an actuator positioned outside the chamber 304 and secured to the adjustable joint 309. The actuator may be configured to adjust the conduit 308 between the first configuration and the second configuration. The actuator may include any aspect of the actuator 232, such as a handle, dial, lever, or grip positioned outside the chamber 304 and secured to the adjustable joint 309. In some embodiments, the actuator 232 may be positioned on the back region of the fluid permeable barrier 302. The actuator outside the chamber 304 may align with the fluid collection region 307a, 307b to which fluid communication is provided within the chamber 304. For example, the actuator may align with or be positioned over the first fluid collection region 307a outside the chamber 304 when the adjustable joint 309 is bent to provide fluid communication between the inlet 317 and the first fluid collection region 307a, and the actuator may align with or be positioned over the second fluid collection region 307b outside the chamber 304 when the adjustable joint 309 is bent to provide fluid communication between the inlet 317 and the second fluid collection region 307b.

The fluid collection device 300 also may include a fluid permeable body (not shown). The fluid permeable body in the fluid collection device 300 may include any aspect of the fluid permeable body 210 described above in relation to the fluid collection device 200. In some embodiments, the fluid permeable body in the fluid collection device 300 may include a slot covering at least a portion of the distal portion 318 of the conduit 308 and configured to allow the distal portion to pivot between the first fluid collection region 307a and the second fluid collection region 307b. In some embodiments, the fluid permeable body in the fluid collection device 300 terminates proximate to the adjustable joint 309 such that a slot is absent from the fluid permeable body. In some embodiments, the distal portion 318 of the conduit 308 is at least partially covered or surrounded by the fluid permeable body and the fluid permeable body may pivot with the distal portion 318 between fluid collection regions 307a, 307b.

In some embodiments, the distal portion 318 of the conduit 308 may be weighted such that the inlet 317 and the distal portion 318 of the conduit 308 falls to the lowest fluid collection region of the fluid collection regions 307a, 307b due to gravity and without assistance or manipulation from the user or caregiver. For example, the fluid collection device 300 may include a ball that pulls the distal portion 318 of the conduit 308 down to the lowest fluid collection region of the fluid collection regions 307a, 307b. In these and other embodiments, the actuator may be absent from the fluid collection device.

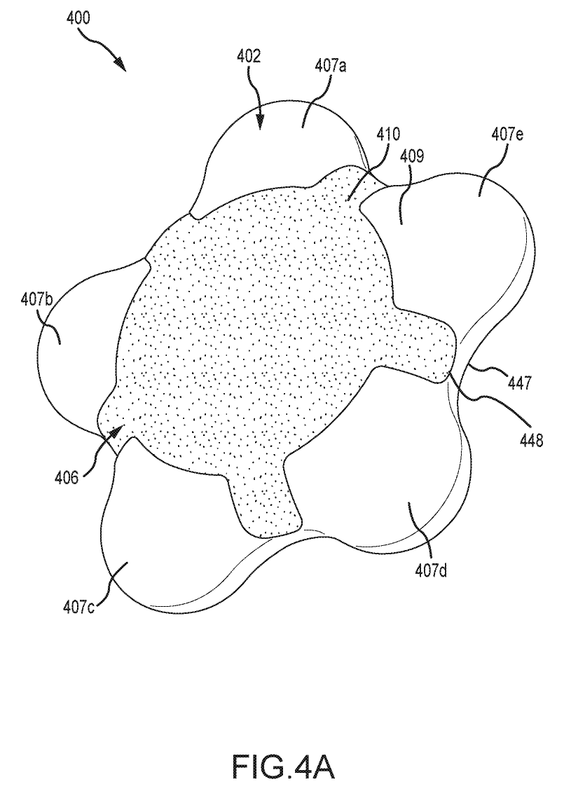

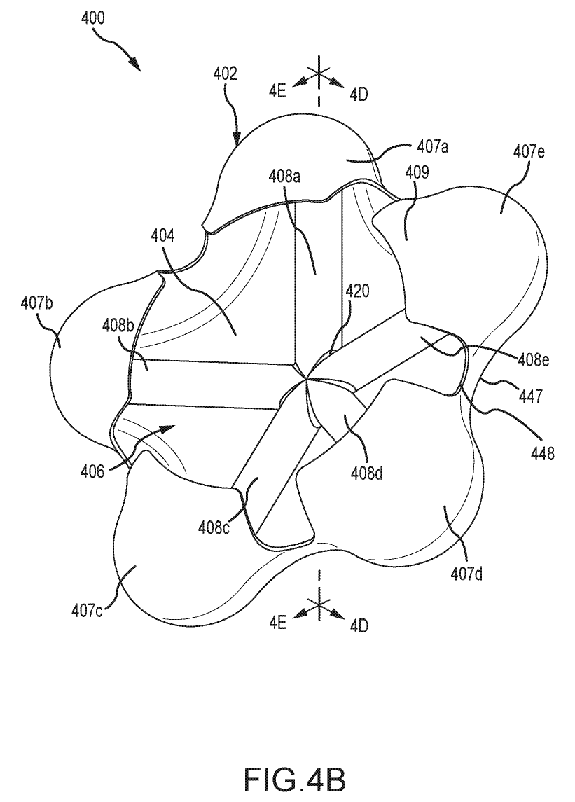

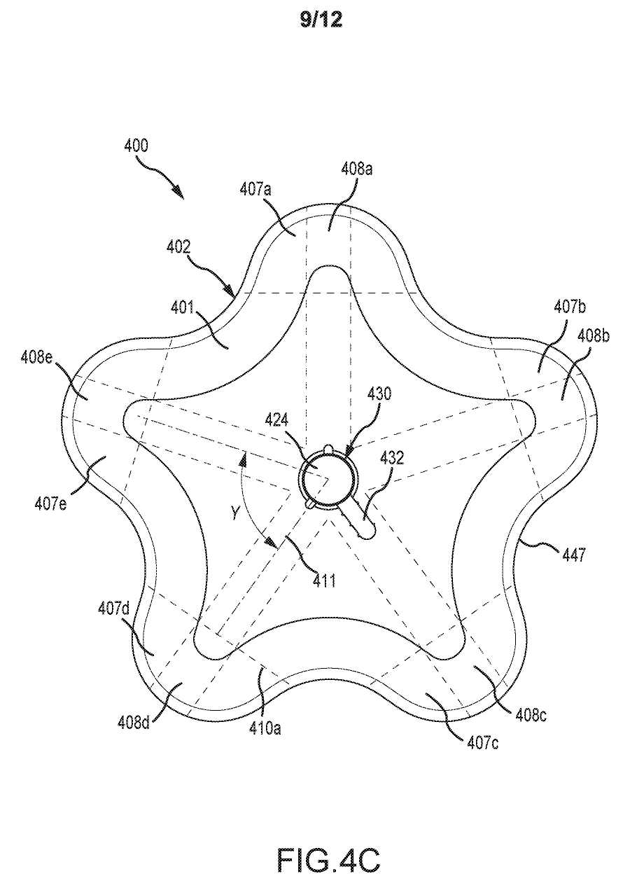

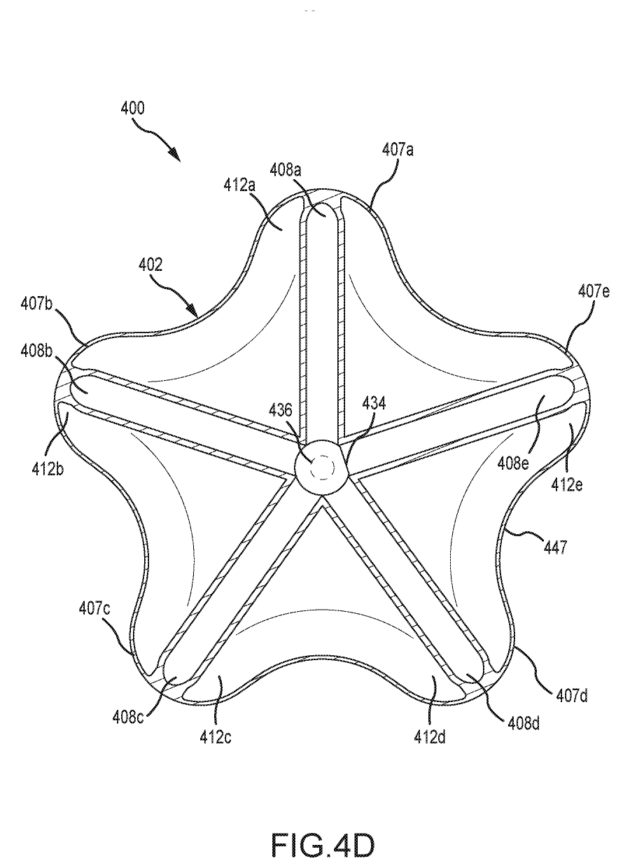

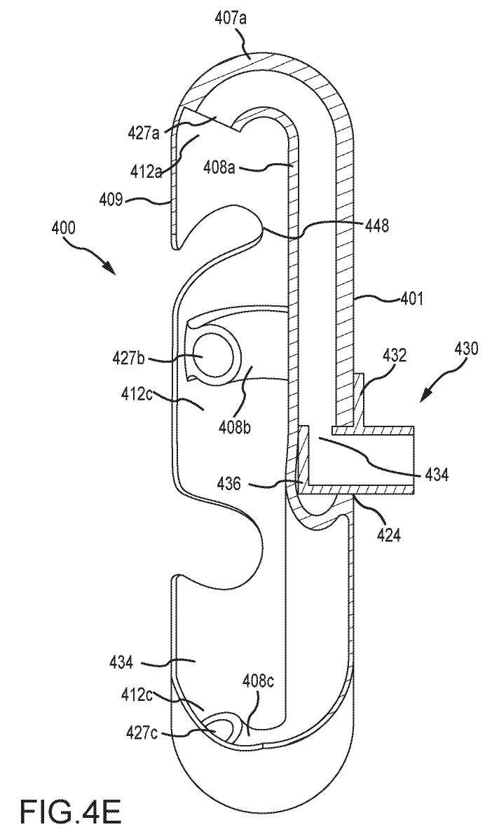

Turning to FIGS. 4A-4E, a fluid collection device 400 may include three or more (e.g., five) fluid collection regions 407a-e each angled relative to one or more adjacent fluid collection regions of the three or more fluid collection regions 407a-e. Unless otherwise noted, the fluid collection device 400 may include any aspect of the fluid collection devices 200, 300. FIG. 4A is a front isometric view of the fluid collection device 400, FIG. 4B is a front isometric view of the fluid collection device 400 with the fluid permeable body 410 removed, FIG. 4C is a rear view of the fluid collection device 400, FIG. 4D is a cross-sectional view of the fluid collection device 400 taken along plane 4D-4D of FIG. 4B, and FIG. 4E is a cross-sectional view of the fluid collection device 400 taken along plane 4E-4E of FIG. 4B

.

The fluid collection device 400 may include a fluid impermeable barrier 402. The impermeable barrier may include any of the materials described above in relation to the fluid impermeable barrier 202. The fluid impermeable barrier 402 may at least partially define a chamber 404, an opening 406 in fluid communication with the chamber 404, an aperture 424, and the three or more fluid collection regions 407a-e each angled relative to adjacent fluid collection regions 407a-e. The fluid impermeable barrier also may include a front region 409 defining the opening 406 and a back region 401 distal to the front region 409 and defining the aperture 424. In some embodiments, the aperture 424 is positioned on a substantially central axis of the fluid impermeable barrier 402.

The fluid collection device 400 includes five fluid collection regions 407a-e and is shaped generally as pentagram having rounded vertices. In some embodiments, the fluid collection device may include three fluid collection regions and be shaped as a three-pointed star having rounded vertices, may include four fluid collection regions and be shaped generally as a four-pointed star having rounded vertices, may include six fluid collection regions and be shaped generally as a hexagram having rounded vertices, may include seven fluid collection regions and be shaped as a heptagram having rounded vertices, may include eight fluid collection regions and be shaped generally as a octogram having rounded vertices, and so on. Each of the fluid collection regions 407a-e may be positioned at a different vertex of the fluid collection device 400.

Each of the fluid collection regions 407a-e may be angled substantially equal to adjacent fluid collection regions 407a-e. For example, turning to FIG. 4C, each of the fluid collection regions 407a-e and/or conduits 408a-e may include an approximate center or axis 411. The center or axis 411 of each fluid collection region 407a-e and/or conduit 408a-e may be angled Y degrees from the center or axis 411 of the adjacent fluid collection region 407a-e and/or conduit 408a-e. In embodiments having five fluid collection regions 407a-e, each of the fluid collection regions 407a-e may be angled approximately 72 degrees from adjacent fluid collection regions 407a-e. In embodiments having three fluid collection regions, each of the fluid collection regions may be angled approximately 120 degrees from adjacent fluid collection regions. In embodiments having four fluid collection regions, each of the fluid collection regions may be angled approximately 90 degrees from adjacent fluid collection regions. In embodiments having six fluid collection regions, each of the fluid collection regions may be angled approximately 60 degrees from adjacent fluid collection regions. In embodiments having seven fluid collection regions, each of the fluid collection regions may be angled approximately 51.4 degrees from adjacent fluid collection regions. In embodiments having eight fluid collection regions, each of the fluid collection regions may be angled approximately 45 degrees from adjacent fluid collection regions.

In some embodiments, the fluid impermeable barrier 402 may include a recessed perimeter 448 on the front region 409 defining a portion of the opening 406 between each of the fluid collection regions 407a-e. The fluid impermeable barrier 402 may include an outer periphery having a concave region 447 between each of the fluid collection regions 407a-e such that the fluid collection regions 407a-e form vertices on the shape or profile of the fluid collection device 400. Each of the fluid collection regions 407a-e of the fluid impermeable barrier 402 may at least partially define a reservoir 412a-e (e.g. sump) in the chamber 404 for fluid collection or storage. In some embodiments, the reservoirs 412a-e may be an at least partially (e.g., entirely) unoccupied portion of the chamber 404 and may be at least partially (e.g., entirely) void of other material. In some embodiments, the reservoirs 412a-e are defined at least partially by a terminating end 410a the fluid permeable body 410 (shown in FIG. 4C) and the fluid collection regions 407a-e of the fluid impermeable barrier 402. The reservoirs 412a-e may be position in the fluid collection regions 407a-e between the concave regions 447 of the outer periphery of the fluid impermeable barrier 402.

The fluid collection device 400 also may include three or more conduits 408a-e. In some embodiments, the fluid collection device 400 includes one conduit for each fluid collection region 407a-e. For example, the fluid collection device 400 includes five conduits 408a-e each having an inlet 427 positioned in or proximate to the reservoir 412a-e of a different fluid collection region 407a-e. FIG. 4E , for example, shows the inlets 427a-c of the conduits 408a-c positioned, respectively, in the reservoirs 412a-c of the fluid collection regions 407a-e. The inlet 427 may be recessed within the fluid permeable body 410 such that the conduits 408a-e do not protrude from the fluid permeable body 410 in the reservoirs 412a-e. The conduits 408a-e may be configured to provide fluid communication between the aperture 424 and the fluid collection regions 407a-e via the inlets 427 on the conduits 408a-e. In some embodiments, the fluid collection device 400 includes a manifold 420 in fluid communication with or through the aperture 424 and from which the conduits 408a-e extend towards the fluid collection regions 407a-e. The manifold 420 may be positioned on the central axis of the fluid collection device 400 and may be in fluid communication with the aperture 424. In some embodiments, the manifold 420 may be integrally formed with the conduits 408a-e. In some embodiments, the manifold 420 and the conduits 408a-e are integrally formed (e.g. molded) with the fluid impermeable barrier 402. In some embodiments, the conduits 408a-e may be detachably secured or securable to the manifold 420, and the manifold 420 may integrally formed with (e.g. molded) the fluid impermeable barrier 402 or detachably secured or securable to the fluid impermeable barrier 402.

In some embodiments the conduits 408a-e are positioned at least proximate to (e.g. secured to or formed on) an inner surface of the back region 409 of the fluid impermeable barrier 402 and extend from the manifold 420 to one of the fluid collection regions 407a-e. Within the fluid collection regions 407a-e, the distal portions of the conduits 408a-e may curl or bend at extend towards the opening 406 such that the inlets 427 of the conduits 408a-e are positioned proximate to the front region 409 of the fluid impermeable barrier 402. The inlets 427 of the conduits 408a-e may be positioned within the reservoirs 412a-e and may be at least partially spaced from the terminating end 410a of the fluid permeable body 410.

In some embodiments, the inlets 427 of the conduits 408a-e may be selectively in fluid communication with the aperture 424 and/or a conduit (not shown) secured to the fluid collection device 400 and providing suction to the fluid collection device 400. For example, the fluid collection device 400 may include a valve 430 configured to selectively allow fluid communication between the aperture 424 and the inlet 427 on one of the conduits 408a-e, but preventing fluid communication between the inlets 427 on the remaining conduits 408a-e. At least a portion of the valve 430 may be valve positioned at least partially within the manifold 220 between at least a portion of the conduits 408a-e and the aperture 424. The valve 430 may be selectively adjustable between a first position, a second position, and at least a third position. In the fluid collection device 400, the valve 430 is selectively adjustable between a first position, a second position, a third position, a fourth position, and a fifth position. In the first position, the valve 430 may be configured to provide fluid communication between the first inlet 427a on the first conduit 408a and the aperture 424 while preventing fluid communication between the aperture 424 and the second inlet 427b on the second conduit 408b, the third inlet 427c on the third conduit 408c, the fourth inlet on the fourth conduit 408d, and the fifth inlet on the fifth conduit 408e. In the second position, the valve 430 may be configured to provide fluid communication between the second inlet 427b on the second conduit 408b and the aperture 424 while preventing fluid communication between the aperture 424 and the first inlet 427a on the first conduit 408a, the third inlet 427c on the third conduit 408c, the fourth inlet on the fourth conduit 408d, and the fifth inlet on the fifth conduit 408e. In the third position, the valve 430 may be configured to provide fluid communication between the third inlet 427c on the third conduit 408c and the aperture 424 while preventing fluid communication between the aperture 424 and the first inlet 427a on the first conduit 408a, the second inlet 427b on the second conduit 408b, the fourth inlet on the fourth conduit 408d, and the fifth inlet on the fifth conduit 408e, and so on.

In some embodiments, the valve 430 includes ball, cylinder, or other rotatable element 436 selectively rotatable within the manifold 220. The rotatable element 436 may include one or more opening 434 or fluid paths extending therethrough and arranged to selectively provide fluid communication between the aperture 424 and the inlet 427 of only one of the conduits 408a-e. For example, the rotatable element 436 may be selectively rotatable between a first position, a second position, and at least a third position to provide the fluid communication described above in the first position, the second third position, and at least the third position of the valve. In embodiments of the fluid collection device 400 having three, four, six, seven, eight or more fluid collection regions 407a-e, the valve 430 may be configured to selectively provide fluid communication between the aperture 424 and the inlet on only one of the conduits 408a-e in only one of the fluid collection regions 407a-e. In some embodiments, the valve 430 may include an additional position that allows fluid communication between the aperture 424 and the inlets 427 on only two adjacent conduits of the conduits 408a-e in only two adjacent fluid collection regions of the fluid collection regions 407a-e.

The fluid collection device 400 also may include an actuator 432 (e.g. , handle, dial, lever, or grip) positioned outside the chamber 404 and secured to the valve 430. The actuator 432 is configured to adjust the valve 430 between the first position, the second position, and at least the third position. In some embodiments, the actuator 432 may be positioned on the back region 401 of the fluid permeable barrier 402. The actuator 432 outside the chamber 404 may align with the fluid collection region 407a-e to which fluid communication is provided in the chamber 404. For example, the actuator 432 may align with or be positioned over the first fluid collection region 407a outside the chamber 404 when the valve 432 provides fluid communication with the first inlet 427a of the first conduit 408a in the first reservoir 412a in the chamber 404, and the actuator 432 may align with or be positioned over the second fluid collection region 407b outside the chamber 404 when the valve 430 provides fluid communication with the second inlet 427b of the second conduit 408b in the second reservoir 412b in the chamber 404, and so on.

In use, the fluid collection device 400 may be positioned on the user with the opening 406 at least proximate to (e.g., positioned against or adjacent to) the region of the user from which fluid is to be collected. Depending on the position of the user and/or the fluid collection device 400, one of the fluid collection regions 407a-e may be positioned lower than the other fluid collection regions 407-ae such that fluid collected flows to the lower fluid collection region. The user or a caregiver may then adjust the actuator 432 to rotate the valve 430 and provide fluid communication between the aperture 424 and the inlet of the conduit in the lower fluid collection region of the fluid collection regions 407a-e while preventing fluid communication between the aperture 424 and the inlets in the higher fluid collection regions of the fluid collection regions 407a-e. A negative pressure or vacuum may then draw fluid in the reservoir of the lower fluid collection region through the inlet in the lower fluid collection region, into the conduit and out of the fluid collection device 400 through the aperture 424. This configuration may allow the fluid collection device 400 to be more efficiently operable in a variety of positions and on a variety of locations on the user.

As noted above, the fluid collection device 400 also includes the fluid permeable body 410 positioned within the chamber 404 and extending at least partially across the opening 406. The fluid permeable body 410 may be shaped generally complementary to the shape of the chamber 404 of the fluid impermeable barrier 402. In some embodiments, the fluid permeable body 410 fills substantially all of the chamber 404, except for the reservoirs 412a-e. For example, the fluid permeable body 210 may include a generally pentagonal profile for a fluid collection device 400 having five fluid collection regions 407a-e. In some embodiments, the fluid permeable body 410 fills substantially all of the chamber 404, including the reservoir 412a-e. The fluid permeable body 410 may include any aspect of the fluid permeable body 210 described above.

In some embodiments, the fluid collection device 400 may include the fluid impermeable barrier 402 and fluid permeable body shown in FIGS. 4A-4E , but the fluid collection device may include a single conduit and single inlet rather than three or more conduits each having an inlet. In this and other embodiments, the single conduit may be rotatable within chamber 404 to position the inlet of the conduit at least proximate to one of the fluid collection regions 407a-e. For example, the conduit may extend through the aperture and towards the outer periphery of the fluid impermeable barrier 402. The actuator 432 may be secured to the conduit such that rotation of the actuator 432 correspondingly rotates the conduit within the chamber 404.

In some embodiments, the entire fluid collection device 400 may be disc- or sector-shaped and include a rotating, internal conduit that, responsive to gravity, rotates around an axis of the fluid collection device such that the inlet of the internal conduit rotates or falls to the low point in the chamber 404 of the fluid collection device 400. For example, the internal conduit may be weighted with a spherical weight such as a bearing or marble. In some embodiments, the fluid collection device 400 may include a ball bearing assembly that replaces the manifold 420 and/or the valve 430, with the internal conduit extending from the ball bearing assembly. A weight may be secured to a distal end region of the internal conduit, proximate to the inlet of the internal conduit. The weight may pull the distal end region of the internal conduit to the low point of the fluid collection device 400, and the ball bearing assembly may be configured to allow the internal conduit to rotate or pivot to position the inlet of the internal conduit at the low point of the fluid collection device 400. The fluid permeable body 410 may be secured to and at least partially surround the internal conduit such that the internal conduit, the fluid permeable body 410, and the weight roll or move in the chamber 404 to the low point in the chamber 404. The internal conduit of the fluid collection device 400 may include multiple inlets at the distal end of the internal conduit. A connector or port may extend through the aperture 424 to provide fluid communication between the internal conduit and a vacuum tube. In some embodiments, the connector rotate in the aperture 424 as the internal conduit rotates.

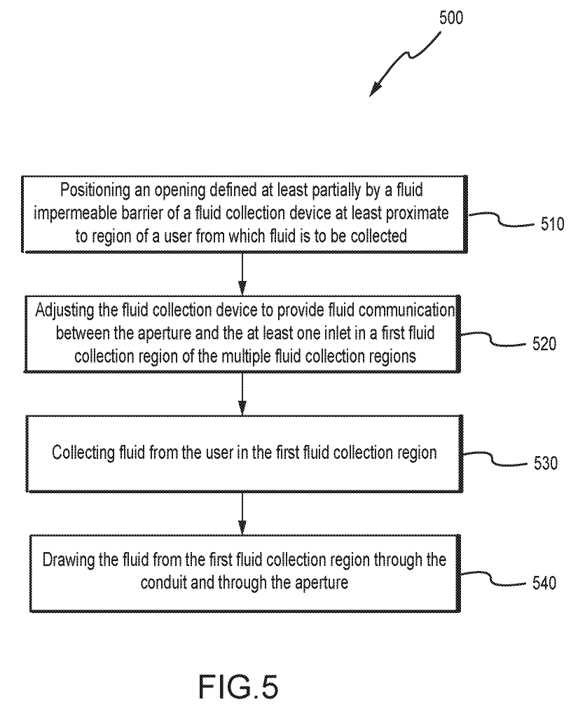

FIG. 5 is a flow diagram of a method 500 of collecting fluid, according to an embodiment. The method 500 includes an act 510 of positioning an opening defined at least partially by a fluid impermeable barrier of a fluid collection device at least proximate to a region of a user from which fluid is to be withdrawn. The method 500 may include an act 520 of adjusting the fluid collection device to provide fluid communication between the aperture and the at least one inlet in a first fluid collection region of the multiple fluid collection regions. Adjusting the fluid collection device in the act 520 also may include inhibiting fluid communication between the aperture and a second fluid collection region of the multiple fluid collection regions, the first fluid collection region being positioned lower than at least the second fluid collection region such that gravity draws fluid to the first fluid collection region. The method 500 also may include an act 530 of collecting fluid from the user in the first fluid collection region. The method 500 also may include an act 540 of drawing the fluid from the first fluid collection region through the conduit and through the aperture.

The method 500 may use the fluid collection system 10 and any of the fluid collection devices 200, 300, 400 describe herein. In some embodiments of the method 500, the fluid impermeable barrier at least partially defines a chamber, an aperture, and multiple fluid collection regions. The fluid collection device may include a fluid permeable body extending at least partially across the opening being positioned at least proximate to the urethral opening, one or more conduits positioned at least partially within the chamber and extending through or in fluid communication with the aperture, and at least one inlet in fluid communication with the one or more conduits and positioned or positionable in each of the multiple fluid collection regions.

In some embodiments of the method 500, the fluid collection device includes embodiments of the fluid collection device 200. The one or more conduits, then, may include at least a proximal conduit and the at least one inlet may include a first inlet positioned within the first fluid collection region and a second inlet positioned within the second fluid collection region. The fluid collection device may include a valve positioned between the proximal conduit and the first inlet and also positioned between the proximal conduit and the second conduit. In these and other embodiments, the act 520 includes adjusting the valve from a second position to a first position that provides fluid communication between the first inlet and the proximal conduit while preventing fluid communication between the second inlet and the proximal conduit, the second position providing fluid communication between the second inlet and the proximal conduit while preventing fluid communication between the proximal conduit and the first inlet.

In some embodiments of the method 500, the fluid collection device may include embodiments of the fluid collection device 300. The one or more conduits, then, may include one conduit extending at least partially through the chamber and the at least one inlet includes an inlet on the one conduit. In these and other embodiments the act 520 may include adjusting the one conduit from a second configuration having the inlet positioned in the second fluid collection region to a first configuration having the inlet positioned in the first fluid collection region.

In some embodiments of the method 500, the fluid collection device may include embodiments of the fluid collection device 400. The multiple fluid collection regions, then, may include a first fluid collection region, a second fluid collection region, and at least a third fluid collection region, each of the first fluid collection region, the second fluid collection region, and the third fluid collection region being angled substantially equal to adjacent fluid collection regions of the first fluid collection region, the second fluid collection region, and the third fluid collection region. The one or more conduits may include multiple conduits each extending from the aperture to a different fluid collection region of the multiple fluid collection regions. The at least one inlet may include multiple inlets, each inlet of the multiple inlets positioned on a different conduit of the multiple conduits and in a different fluid collection region of the multiple fluid collection regions. The multiple conduits include a first conduit including a first inlet of the multiple inlets, a second conduit including a second inlet of the multiple inlets, and at least a third conduit including a third inlet of the multiple inlets. The fluid collection device may include a valve positioned between the aperture and at least a portion of each of the multiple conduits. In these and other embodiments, the act 520 may include adjusting the valve from one of a second position or a third position to a first position. The first position may provide fluid communication between the first inlet and the aperture while preventing fluid communication between (1) the aperture and the second inlet and (2) the aperture and the third inlet. The second position may provide fluid communication between the second inlet and the aperture while preventing fluid communication between (1) the aperture and the first inlet and (2) the aperture and the third inlet. The third position may provide fluid communication between the third inlet and the aperture while preventing fluid communication between (1) the aperture and the first inlet and (2) the aperture and the second inlet.

The acts of the method 500 described above are for illustrative purposes. For example, the acts of the method 500 can be performed in different orders, split into multiple acts, modified, supplemented, or combined. In an embodiment, one or more of the act of the method 500 can be omitted from the method 500. Any of the acts of the method 500 can include using any of the fluid collection devices and systems disclosed herein.

As used herein, the term “about” or “substantially” refers to an allowable variance of the term modified by “about” or “substantially” by ±10% or ±5%. Further, the terms “less than,” “or less,” “greater than,” “more than,” or “or more” include, as an endpoint, the value that is modified by the terms “less than,” “or less,” “greater than,” “more than,” or “or more.”

While various aspects and embodiments have been disclosed herein, other aspects and embodiments are contemplated. The various aspects and embodiments disclosed herein are for purposes of illustration and are not intended to be limiting.

What is claimed is:

1. A fluid collection device, comprising:
a fluid impermeable barrier at least partially defining a chamber, an opening in fluid communication with the chamber, an aperture, and multiple fluid collection regions each angled relative to one or more adjacent fluid collection regions
of the multiple fluid collection regions;
one or more conduits positioned at least partially within the chamber, a conduit of the one or more conduits extending through or in fluid communication with the aperture in the fluid impermeable barrier;
at least one inlet in fluid communication with the one or more conduits and positioned or positionable in each of the multiple fluid collection regions; and
a fluid permeable body positioned within the chamber and extending across at least a portion of the opening,
wherein the fluid collection device is adjustable between (1) a first configuration providing fluid communication between a first fluid collection region of the multiple fluid collection regions and the conduit extending through the aperture while preventing fluid communication between a second fluid collection region of the multiple fluid collection regions and the conduit extending through the aperture and (2) a second configuration providing fluid communication between the second fluid collection region and the conduit extending through the aperture while preventing fluid communication between the first fluid collection region and the conduit extending through the aperture.
